(19) United States
(12) Patent Application Publication
(10) Pub. No.: US 2018/0317529 A1
(43) Pub. Date: Nov. 8, 2018

(54) BEADLETS COMPRISING CAROTENOIDS

(71) Applicant: DSM IP ASSETS B.V., Heerlen (NL)

(72) Inventors: Bernd SCHLEGEL, Basel (CH); Gabriela BADOLATO BÖNISCH, Basel (CH)

(21) Appl. No.: 16/031,800

(22) Filed: Jul. 10, 2018

Related U.S. Application Data
(62) Division of application No. 14/113,007, filed on Jun. 6, 2014, now abandoned.

(30) Foreign Application Priority Data
Apr. 20, 2011 (CH) 00706/11

(51) Int. Cl. A23L 5/44; A61K 31/202; A23K 20/158; A61K 31/015; A61K 31/01; A61K 9/50; A23L 2/58; A23D 7/01; A23D 7/005; A23P 10/35; A23L 33/115; A23L 33/105; A23P 10/20; A23L 33/12; A23L 27/00; A23K 40/10; A23K 20/179; A23K 20/174; A61K 31/122

(52) U.S. Cl. CPC A23L 5/44; A61K 31/122; A23K 20/158; A61K 31/015; A61K 31/01; A61K 9/5063; A23L 2/58; A23D 7/013; A23D 7/0056; A23P 10/35; A23L 33/115; A23L 33/105; A23P 10/20; A23L 33/12; A23L 27/80; A23K 40/10; A23K 20/179; A23K 20/174; A61K 31/202

(57) ABSTRACT

The present invention relates to beadlets comprising at least one carotenoid and matrix material, which comprise wax(es) and/or fat(s) with a drop point of between 30 to 85° C., as well as to the production of such beadlets and to the use of such beadlets in compositions.

### CROSS-REFERENCE TO RELATED APPLICATIONS

This application is a divisional of commonly owned copending U.S. Ser. No. 14/113,007, filed Jun. 6, 2014 (now abandoned), which is the national phase application of International Application No. PCT/EP2012/057238, filed Apr. 20, 2012, which designated the U.S. and claims priority to Swiss Patent Application No. 00706/11, filed Apr. 20, 2011, the entire contents of each of which are hereby incorporated by reference.

### FIELD

The present invention relates to beadlets comprising at least one carotenoid and matrix material, which comprise wax(es) and/or fat(s) with a drop point of between 30 to 85° C., as well as to the production of such beadlets and to the use of such beadlets in compositions.

### BACKGROUND AND SUMMARY

Nowadays there are many forms of formulations available, which comprise carotenoids. Solid, liquid or paste-like formulations are known. Their formulations do have disadvantages. The liquid formulations do have a tendency to get inhomogenously, so that they have to be shaken regularly to avoid that.

The problems with the solid formulations are for example
i) dust issues (not easy to handle and risk of explosion),
ii) the particles stick together.

The goal of the present invention was to find a form of formulation, which is easy to produce and which is also good and safe to handle.

It was found out that when a formulation in the form of beadleta, which comprise at least one carotenoid and a matrix (waxes and/or fats with specific and well defined properties), was produced then a formulation was obtained which does not have the disadvantages as mentioned above.

And furthermore, such beadlets can be produced by using the spray chilled process, which is a mild production procedure. The principle of this process is widely known.

The main advantages of the beadlets according to the present invention are the following
(i) good and fast dispersibilty into an oil (phase); and
(ii) good flowabilty (not sticky, not dusty and easy to dose); this property result in essentially no material loss when transferred from one container to another and a container, wherein the beadlets have been stored can be cleaned easily; and
(iii) The color saturation as well as the color stability of the beadlets in an end-market product (consumer product, which is sold in (grocery) shops) is good (also after pasteurization).

Therefore, the present invention relates to beadlets (I) which comprise
(i) 1 weight-% (wt-%) to 50 wt-%, based on the total weight of the beadlets, of at least one carotenoid, and
(ii) 50 wt-% to 99 wt-%, based on the total weight of the beadlets, of a matrix material comprising at least one wax and/or hydrated fat, characterized in that the matrix material has a drop point of from 30 to 85° C.

It is clear that the percentages always add up to 100%. This requirement applies to all compositions.

Beadlets (comprising fat-soluble substances) and their methods of productions are known from the prior art, for example from US 2006/0115534 and U.S. Pat. No. 4,670,247. These beadlets usually have good storage stability, but the concentration of the fat-soluble substances in such beadlets is low. Usually the content is between 5 to 15 weight-% (wt-%), based on the total weight of the beadlets.

With the present invention it was possible to form non-sticky, non dusty beadlets by using the cold spray drying granulation technology. Furthermore, these beadlets can comprise up to 50 wt-%, based on the total weight of the beadlet, of at least one carotenoid.

### BRIEF DESCRIPTION OF THE DRAWINGS

FIG. 1: CIE L*a*b* and C*h* in coordinate system.

FIG. 2: Colour difference in soft drink application during storage.

FIG. 3: Colour difference in a margarine during storage.

### DETAILED DESCRIPTION

Preferred amounts of at least one carotenoid in the beadlets according to the present invention are 10 wt-%-50 wt-%, 15 wt-%-50 wt-%, 20 wt-%-50 wt-%, 10 wt-%-45 wt-%, 15 wt-%-45 wt-%, 20 wt-%-45 wt-%, 10 wt-%-40 wt-%, 15 wt-%-40 wt-% and 20 wt-%-40 wt-% (all based on the total weight of the beadlets).

As a consequence thereof the preferred amounts of matrix material comprising at least one wax and/or hydrated fat, characterized in that the matrix material has a drop point of from 30 to 85° C., are 50 wt-%-90 wt-%, 50 wt-%-85 wt-%, 50 wt-%-80 wt-%, 55 wt-%-90 wt-%, 55 wt-%-85 wt-%, 55 wt-%-80 wt-%, 60 wt-%-90 wt-%, 60 wt-%-85 wt-% and 60 wt-%-80 wt-% (all based on the total weight of the beadlets).

Therefore a preferred embodiment of the present invention are beadlets (II) which comprise
(i) 10 wt-% - 50 wt-%, based on the total weight of the beadlets, of at least one carotenoid, and
(ii) 50 to 90 wt-%, based on the total weight of the beadlet,s of a matrix material comprising at least one wax and/or hydrated fat, characterized in that the matrix material has a drop point of from 30 to 85° C.

Therefore a preferred embodiment of the present invention are beadlets (III) which comprise
(i) 15 wt-%-50 wt-%, based on the total weight of the beadlets, of at least one carotenoid, and
(ii) 50 wt-%-85 wt-%, based on the total weight of the beadlets, of a matrix material comprising at least one wax and/or hydrated fat, characterized in that the matrix material has a drop point of from 30 to 85° C.

Therefore a preferred embodiment of the present invention are beadlets (IV) which comprise
(i) 20 wt-%-50 wt-%, based on the total weight of the beadlets, of at least one carotenoid, and
(ii) 50 wt-%-80 wt-%, based on the total weight of the beadlets, of a matrix material comprising at least one wax and/or hydrated fat, characterized in that the matrix material has a drop point of from 30 to 85° C.

Therefore a preferred embodiment of the present invention are beadlets (V) which comprise
(i) 10 wt-%-45 wt-%, based on the total weight of the beadlets, of at least one carotenoid, and
(ii) 55 wt-%-90 wt-%, based on the total weight of the beadlets, of a matrix material comprising at least one wax and/or hydrated fat, characterized in that the matrix material has a drop point of from 30 to 85° C.

Therefore a preferred embodiment of the present invention are beadlets (VI) which comprise
(i) 15 wt-%-45 wt-%, based on the total weight of the beadlets, of at least one carotenoid, and
(ii) 55 wt-%-85 wt-%, based on the total weight of the beadlets, of a matrix material comprising at least one wax and/or hydrated fat, characterized in that the matrix material has a drop point of from 30 to 85° C.

Therefore a preferred embodiment of the present invention are beadlets (VII) which comprise
(i) 20 wt-%-45 wt-%, based on the total weight of the beadlets, of at least one carotenoid, and
(ii) 55 wt-%-80 wt-%, based on the total weight of the beadlets, of a matrix material comprising at least one wax and/or hydrated fat, characterized in that the matrix material has a drop point of from 30 to 85° C.

Therefore a preferred embodiment of the present invention are beadlets (VIII) which comprise
(i) 10 wt-%-40 wt-%, based on the total weight of the beadlets, of at least one carotenoid, and
(ii) 60 wt-%-90 wt-%, based on the total weight of the beadlets, of a matrix material comprising at least one wax and/or hydrated fat, characterized in that the matrix material has a drop point of from 30 to 85° C.

Therefore a preferred embodiment of the present invention are beadlets (IX) which comprise
(i) 15 wt-%-40 wt-%, based on the total weight of the beadlets, of at least one carotenoid, and
(ii) 60 wt-%-85 wt-%, based on the total weight of the beadlets, of a matrix material comprising at least one wax and/or hydrated fat, characterized in that the matrix material has a drop point of from 30 to 85° C.

Therefore a preferred embodiment of the present invention are beadlets (X) which comprise
(i) 20 wt-%-40 wt-%, based on the total weight of the beadlets, of at least one carotenoid, and
(ii) 60 wt-%-80 wt-%, based on the total weight of the beadlets, of a matrix material comprising at least one wax and/or hydrated fat, characterized in that the matrix material has a drop point of from 30 to 85° C.

The term “beadlet(s)” as used herein refers to small discrete particles, which have a mean (average) particle size of 50 μm to 1000 μm in diameter. (preferably from 250 μm to 850 μm). The sizes can be smaller or larger. The size of a beadlet can be determined according to well known methods, such as (scanning) electron microscopy. Beadlets are usually nearly spherical. Beadlets contain one or more active ingredients in an encapsulated form.

Therefore a further embodiment of the present invention relates to beadlets (XI), which are beadlets (I), (II), (III), (IV), (V), (VI), (VII), (VIII), (IX) and/or (X), which have mean (average) particle sizes of 50 μm to 1000 μm in diameter. (preferably from 250 μm to 850 μm).

The beadlets according to the present invention comprise at least one carotenoid. The term “carotenoid” as used herein comprises a carotene or structurally related polyene compound which can be used as a colorant for food, beverages, animal feeds, cosmetics or drugs. Examples of such carotenoids are α- or β-carotene, 8′-apo-β-carotenal, 8′-apo-β-carotenoic acid esters such as the ethyl ester, canthaxanthin, astaxanthin, lycopene, lutein, zeaxanthin or crocetin, or mixtures thereof. The preferred carotenoid is β-carotene.

Therefore a preferred embodiment of the present invention relates to beadlets (XII), which are beadlets (I), (II), (III), (IV), (V), (VI), (VII), (VIII), (IX), (X) and/or (XI), wherein the at least one carotenoid is chosen from the group consisting of α- or β-carotene, 8′-apo-β-carotenal, 8′-apo-β-carotenoic acid esters (such as the ethyl ester), canthaxanthin, astaxanthin, lycopene, lutein, zeaxanthin and crocetin.

A more preferred embodiment of the present invention relates to beadlets (XII′), which are beadlets (I), (II), (III), (IV), (V), (VI), (VII), (VIII), (IX), (X) and/or (XI), wherein the carotenoid is β-carotene.

It is clear that the percentages always add up to 100%.

The beadlets according to the present invention comprise at least one wax and/or hydrated fat with a drop point of from 30 to 85° C., preferably 40 to 70° C. Waxes in the context of the present invention are organic compounds that characteristically consist of a long alkyl chains. Natural waxes (plant, animal) are typically esters of fatty acids and long chain alcohols. Synthetic waxes are long-chain hydrocarbons lacking functional groups.

Fats consist of a wide group of compounds that are generally soluble in organic solvents and largely insoluble in water. Hydrated fats (or saturated fats) in the context of the present invention are generally triesters of glycerol and fatty acids. Fatty acids are chains of carbon and hydrogen atoms, with a carboxylic acid group at one end. Such fats can have natural or synthetic origin. It is possible to hydrate a (poly)unsaturated fat to obtain a hydrated (saturated) fat.

The matrix which comprises at least one wax and/or fat has a drop point of 30 to 85° C. The drop point of a material is that temperature (in ° C.) when the material begins to melt under standardized conditions. The material is heated so long until it changes the state of matter from solid to liquid. The drop point is the temperature when the first drop is released from the material. The determination of the drop point (Tropfpunkt) is carried out as described in the standard norm DIN ISO 2176.

Preferred examples of waxes and hydrated fats suitable for the present invention are glycerin monostearate, carnauba wax, candelilla wax, palmitic acid, stearic acid and hydrated rapeseed oil. These compounds can be used as such or as mixtures.

Therefore a preferred embodiment of the present invention relates to beadlets (XIII), which are beadlets (I), (II), (III), (IV), (V), (VI), (VII), (VIII), (IX), (X), (XI), (XII) and/or (XIII wherein the at least one wax and/or fat having a drop point of 30 to 85° C. (preferably 40 to 70° C.), is chosen from the group consisting of glycerin monostearate, carnauba wax, candelilla wax, palmitic acid, stearic acid and hydrated rapeseed oil.

The beadlets can comprise further auxiliary agents (auxiliaries). Depending for what the beadlets are used, the auxiliary agent(s) can vary. These auxiliary agents can be useful for the formulation by further improving its properties, such as physical stability, storage stability, visual perception, etc. Auxiliaries can also be useful for the application in the food or feed product by improving the property of these compositions, physical stability, storage stability, visual perception, controlled release in the GI-tract, pH control, oxidation resistant, etc. The concentration of these auxiliaries can vary, depending on the use of these auxiliaries. These auxiliary agents are usually present in an amount of 0 wt-% to 5 wt-%, based on the total weight of the beadlets.

The beadlets can optionally comprise for example antioxidants.

Antioxidants prevent oxidation of the active ingredients, thus preserving the desired properties of the actives, such as biological activity, color and/or color intensity. Typical antioxidants are vitamin E (tocopherol), vitamin C, ascorbyl palmitate, 2,6-di-tert-butyl-p-cresol (butylated hydroxytoluene or BHT), butylated hydroxyanisole (BHA), ethoxyquin (EMQ), and others. These compounds can be used as such or as mixtures. The beadlets of the present invention comprise 0 to 5 wt-%, based on the total weight of the beadlets, of at least one antioxidant. Therefore a preferred embodiment of the present invention relates to beadlets (XIV), which are beadlets (I), (II), (III), (IV), (V), (VI), (VII), (VIII), (IX), (X), (XI), (XII), (XII′) and/or (XIII), wherein the beadlets comprise 0 wt-% to 5 wt-%, based on the total weight of the beadlets, of at least one auxiliary agent.

A more preferred embodiment of the present invention relates to beadlets (XIV′), which are beadlets (XIV), wherein the beadlets comprise 0 wt-% to 5 wt-%, based on the total weight of the beadlets, of at least one antioxidant.

An even more preferred embodiment of the present invention relates to beadlets (XIV″), which are beadlets (XIV), wherein the beadlets comprise 0 wt-% to 5 wt-%, based on the total weight of the beadlets, of at least one antioxidant chosen from the group consisting of vitamin E (tocopherol), vitamin C, ascorbyl palmitate, 2,6-di-tert-butyl-p-cresol (butylated hydroxytoluene or BHT), butylated hydroxyanisole (BHA) and ethoxyquin (EMQ).

An especially preferred embodiment of the present invention relates to beadlets (XIV″), which are beadlets (XIV), wherein the beadlets comprise 0 wt-% to 5 wt-%, based on the total weight of the beadlets, of at least one antioxidant, which vitamin E (tocopherol).

The beadlets are usually produced by using the spray chilled technology or spray cooling technology. This technology is widely known in the field spray drying. It is described for example in trends in Food Science & Technology 15 (2004), 330-347.

The steps of spray chilled or spray cooling technology (process) are:
1) mixing the all ingredients (incl. the wax and/or hydrated fat)m, and
2) atomizing into a chamber, where it is contacted with an air stream which is cool enough to case the droplets to solidify.

It is also preferred that the mixture before atomizing, is grinded. The grinding step can be carried out by various types of mills, i.e. a colloid mill or a ball mill.

The present invention also relates to the production of beadlets (I), (II), (III), (IV), (V), (VI), (VII), (VIII), (IX), (X), (XI), (XII), (XII′), (XIII), (XIV), (XIV′), (XIV″) and/or (XIV′″) by using the spray chilled process or the spray cooling process.

The present invention also relates to the production of beadlets (I), (II), (III), (IV), (V), (VI), (VII), (VIII), (IX), (X), (XI), (XII), (XII′), (XIII), (XIV), (XIV′), (XIV″) and/or (XIV′″) using the spray chilled process or the spray cooling process, wherein the process comprising the following steps:
1) mixing the all ingredients (incl. the wax and/or hydrated fat), and
2) atomizing into a chamber, where it is contacted with an air stream which is cool enough to case the droplets to solidify.

Preferably, the present invention also relates to the production of beadlets (I), (II), (III), (IV), (V), (VI), (VII), (VIII), (IX), (X), (XI), (XII), (XII′), (XIII), (XIV), (XIV′), (XIV″) and/or (XIV″′) using the spray chilled process or spray cooling process, wherein the process comprising the following steps:
1) mixing the all ingredients (incl. the wax and/or hydrated fat), and
2) grinding the mixture of the ingredients (preferably by a colloid mill or a ball mill), and
3) atomizing into a chamber, where it is contacted with an air stream which is cool enough to case the droplets to solidify.

The carotenoid particles (inside the beadlet) do usually have a size (d0.9) of below 20 μm. When a ball mill is used then the carotenoid particles do usually have a size (d 0.9) of below 10 μm.

Therefore, the present invention also relates to beadlets (XV), which are beadlets (I), (II), (III), (IV), (V), (VI), (VII), (VIII), (IX), (X), (XI), (XII), (XII′), (XIII), (XIV), (XIV′), (XIV″) and/or (XIV′″), wherein the carotenoid particles (inside the beadlets) have a size (d0.9) of below 20 μm.

Therefore, the present invention also relates to beadlets (XV′), which are beadlets (I), (II), (III), (IV), (V), (VI), (VII), (VIII), (IX), (X), (XI), (XII), (XII′), (XIII), (XIV), (XIV′), (XIV″) and/or (XIV′″), wherein the carotenoid particles (inside the beadlets) have a size (d0.9) of below 10 μm.

The beadlets according to the present invention can be used in many fields of applications. It can be used in food, feed and personal care products. Preferred is the use in food products, very preferred it the use of beadlets as described above in margarines and in beverages (such as soft drinks). The amount, which is used in such products, depends heavily on the product as well as of the color shade which is desired.

The beadlets (I), (II), (III), (IV), (V), (VI), (VII), (VIII), (IX), (X), (XI), (XII), (XII′), (XIII), (XIV), (XIV′), (XIV″), (XIV″′), (XV) and/or (XV′) are used in the production of food, feed and personal care products; preferably in the production of food products, more preferably in the production of (soft) drinks and margarines.

It is clear that all other commonly known ingredients for producing food, feed and personal care products are also used in the process.

Therefore the present invention also relates to a process of production of food, feed and personal care products, wherein beadlets (I), (II), (III), (IV), (V), (VI), (VII), (VIII), (IX), (X), (XI), (XII), (XII′), (XIII), (XIV), (XIV′), (XIV″), (XIV″′), (XV) and/or (XV′) are used.

As already stated above the amount of the beadlets (I), (II), (III), (IV), (V), (VI), (VII), (VIII), (IX), (X), (XI), (XII), (XII′), (XIII), (XIV), (XIV′), (XIV″), (XIV″′), (XV) and/or (XV′) used in the production of food, feed and personal care products depends on the product. Usually the amount of the at least one carotenoid in the food, feed and personal care product is 1 to 12 ppm.

In a soft drink the amount of the at least one carotenoid is 1 to 12 ppm.

In a margarine the amount of the at least one carotenoid is 1 to 12 ppm.

Therefore the present invention also relates to a process of production of food, feed and personal care products, wherein beadlets (I), (II), (III), (IV), (V), (VI), (VII), (VIII), (IX), (X), (XI), (XII), (XIII (XIII), (XIV), (XIV′), (XIV″), (XIV′″), (XV) and/or (XV′) are used and wherein the amount of at least one carotenoid in the food, feed and personal care products is 1 ppm to 12 ppm.

A further embodiment of the present relates to food, feed and personal care products obtained from a process as described above.

As mentioned above the advantages of the beadlets are also that they allow to producing end-market products (consumer products, which are sold i.e. in (grocery) shops), which have good color saturation as well as good color stability (during storage).

All the color measurements of the present patent application are carried out with a colorimeter (Hunter Lab Ultra Scan Pro) which can other than a spectrophotometer express colour values according to the psychophysical perception of colour by human eye.

Colour measurements are carried out after CIE guidelines (Commission International d'Eclairage). Values can be expressed either as planar coordinates L*a*b* with L* being the measuring value for lightness, with a*being the value on red-green-axis and with b* being the value on the yellow-blue-axis.

The colour can also be expressed in cylindrical coordinates C* and h with C* (chroma) being a value for colour saturation and with h (hue) being the angle-value for colour shade. With the cylindrical coordinates easy comparisons between various product colours can be made (FIG. 1).

For the measurements of the samples of the present invention the following instrument settings have been used:
Colour scale : CIE L*a*b*/L*C*h*
Light source definition: D65 daylight equivalent
Geometry: Diffuse/8°
Wavelengths: scan 350 to 1050 nm in 5 nm optical resolution
Sample measurement area diameter: 19 mm (large)
Calibration mode: Transmission (for soft drink) and reflection (for margarine)/white tile

Colour difference was calculated using following equation:

DE*=√(ΔL)²+(Δa)²+(Δb)², wherein

L: Lightness

A: Red value and

B: Yellow value

A DE*-value of <10 is desired to have a good (stable) product.

The invention is illustrated by the following Examples. All temperatures are given in ° C. and all parts and percentages are related to the weight.

### Process Examples

The first two examples are related to the process of production of the beadlets according to the present invention

### Example 1

66.3 g glyeridmonostearate (GMS) have been put into a vessel and heated up to 85° C. under inert gas. 0.7 g dl-α-tocopherol has been added under stirring to the melted GMS. Afterwards 33.g of crystalline β-carotene was added to the reaction mixture. The mixture is stirred and afterward at 85° C. grinded by using a colloid mill.

Afterwards the reaction mixture was spray dried by using the spray chilling technology (the air had a temperature of 5° C.). Non-sticky and non-dusty beadlets with a particle size (d 0.5) of 384 μm have been obtained. The particle size (d 0.9) of the carotenoid particles (inside the beadlets) was 15 μm.

### Example 2

66.3 g glyeridmonostearate (GMS) have been put into a vessel and heated up to 85° C. under inert gas. 0.7 g dl-α-tocopherol has been added under stirring to the melted GMS. Afterwards 33.g of crystalline β-carotene was added to the reaction mixture. The mixture is stirred and afterward at 85° C. grinded by using a ball mill.

Afterwards the reaction mixture was spray dried by using the spray chilling technology (the air had a temperature of 5° C.). Non-sticky and non-dusty beadlets with a particle size (d 0.5) of 341 μm have been obtained. The particle size (d 0.9) of the carotenoid particles (inside the beadlets) was 9 μm.

### Application Examples

The following examples are related to the use of the beadlets according to the present invention in the production food products

### Example 3: Process of Production of a Flavour β-Carotene Emulsion (1.2 g/l β-Carotene)

In a first step 50 g of ester gum was dissolved in 50 g of orange oil under stirring at 70° C.

Afterwards, in a second step, 80 g of the solution of step 1 was mixed with 4 g of the beadlets of Example 1 in a beaker. The beaker was placed in a water bath (80° C.) and the mixture was stirred until the beadlets were dispersed completely.

In a second beaker, 336 g of a sugar syrup (having 64° Brix) was mixed with 560 g of a gum acacia solution (40 wt-%, based on the weight of this solution, of gum acacia and 60 wt-% of water). 20 g of ascorbic acid was added to this mixture under stirring at 50° C.

Afterwards both mixtures were combined under stirring and then pre-emulsified at 50° C. using a rotor stator. Finally the pre-emulsified mixture was homogenised using a high pressure homogeniser for 2 minutes at 200/50 bar.

### Example 4: Soft Drink (3 ppm β-Carotene Content)

In a 1 l volumetric flask 0.2 g potassium sorbate was dissolved in 38.4 g water. To this solution 156.2 g sugar syrup (having 64° Brix), 0.2 g ascorbic acid and 5 g of a 50% solution of citric acid (50 wt-%, based on the weight of this solution, of citric acid and 50 wt-% of water) were added subsequently under stirring.

Afterwards 2.5 g of the flavour β-carotene emulsion of Example 3 were added and then water was added to fill up the volumetric flask to 1 litre.

The so obtained yellowish soft drink was filled into glass bottles (closed by bottle cap, crown cap) and pasteurized in a water bath at 80° C. for 1 min.

The so obtained yellowish soft drink had the following properties:

Colour saturation (C*)=43.1

Colour shade (h)=83.6°

These soft drinks in the glass bottles were stored at room temperature.

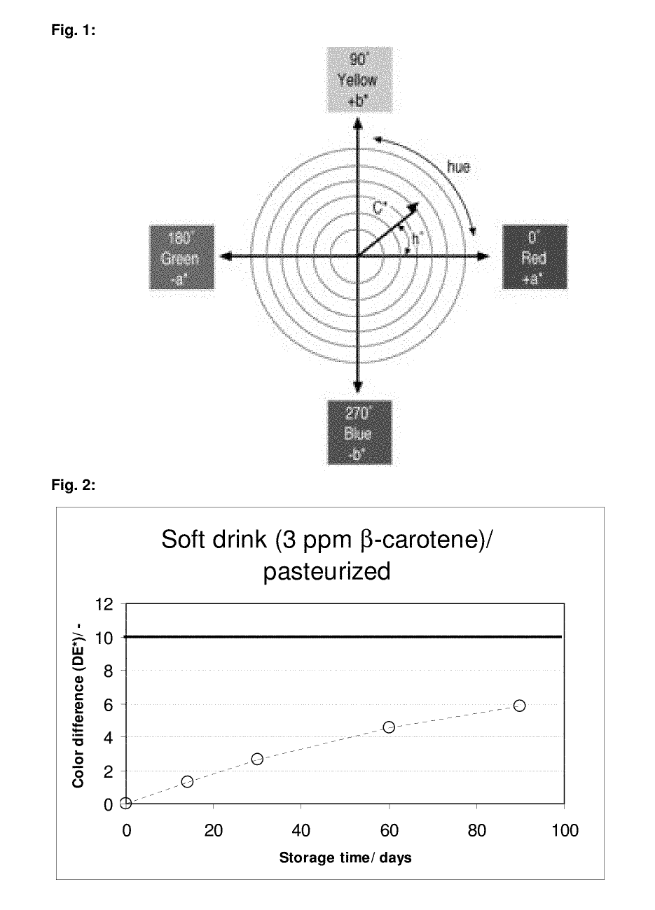

The soft drinks showed a very good performance related to the colour stability during the storage time. The DE* after 90 days was less than 6. (s. FIG. 2).

### Example 5: Margarine (6 ppm β-Carotene Content)

1 g of the beadlets of Example 1 was dispersed in 99 g of sunflower oil (at 60° C.) under stirring until the beadlets were dispersed completely (first solution).

A second solution (the water phase) was prepared by dissolving 15 g milk powder and 5 g NaCl in 152 g of water.

A third solution (the oil phase) was prepared by warming (60° C.) the coconut and then adding 4 g of Lamemul® K 2001 S (from Cognis; this is 90% monoglycerides; spray-chilled) and 2 g of soya lecithin (Yellothin 100 IP soy lecithin from Sternchemie, Germany). Afterwards 320 g of sunflower oil was added as well as the first solution. The mixture was then heated 50° C.

Then the water phase was added slowly by using a rotor stator (high speed) to this mixture and the obtained product was treated additionally for 1 min with the rotor stator at high speed.

The so obtained margarine was put into an ice machine and cooled down to 5° C. Then the margarine was filled into typical containers and stored in a fridge at 4° C.

The so obtained margarine had the following properties:

Colour saturation (C*)=37.6

Colour shade (h)=78.7°

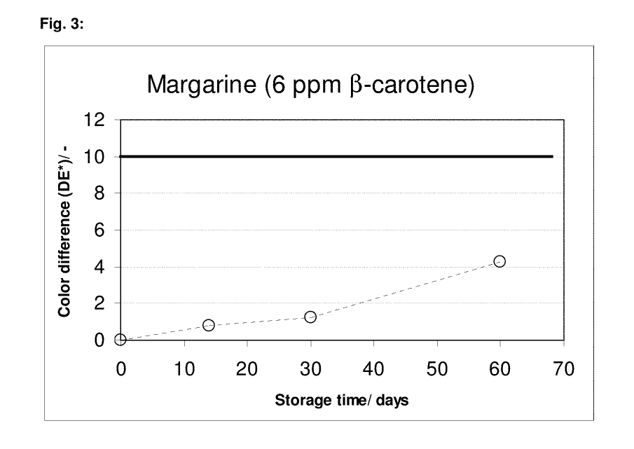

The margarines showed a very good performance related to the colour stability during the storage time. The DE* after 60 days was less than 5. (s. FIG. 3).

1. A process for producing beadlets comprising:
(1) forming an ingredient mixture comprised of:
(i) 1 wt-%-50 wt-%, based on the total weight of the beadlets, of crystalline β-carotene particles; and
(ii) 50-99 wt-%, based on the total weight of the beadlets, of a matrix material having a drop point of from 30 to 85° C. which is at least one selected from the group consisting of lycerinmonostearate, carnauba wax, candelilla wax, palmitic acid, stearic acid and hydrated rapeseed oil;
(2) subjecting the ingredient mixture to a spray chilling or spray cooling process by atomizing the ingredient mixture into a chamber to form droplets of the ingredient mixture, and
(3) contacting the droplets of the ingredient mixture in the chamber with an air stream which is sufficiently cool to solidify the ingredient droplets and form the beadlets.

2. The process according to claim 1, which further comprises grinding the ingredient mixture before atomizing.

3. The process according to claim 1, wherein steps (b) and (c) are practiced to form beadlets having a mean particle size of 50 μm to 1000 μm in diameter.

4. The process according to claim 3, wherein the particle size of the beadlets is from 250 μm to 850 μm.

5. The process according to claim 1, wherein prior to step (2) the process further comprises grinding the ingredient mixture.

6. The process according to claim 5, wherein the step of grinding is practiced with a colloid mill or a ball mill.

7. The process according to claim 5, wherein the β-carotene particles have a size (d0.9) of below 10 μm.

8. The process according to claim 1, wherein the β-carotene particles have a size (d0.9) of below 20 μm.

9. The process according to claim 1, wherein the beadlets have a color difference (DE*) of less than 10 as calculated by the equation:
DE*=√(ΔL)²+(Δa)²+(Δb)²,
wherein L is Lightness, A is Red value and B is Yellow value.

10. The process according to claim 1, wherein step (3) is practiced by contacting the droplets of the ingredient mixture with chilled air at a temperature of about 5° C.

12. A process for producing a food, feed or personal care product, wherein the process comprises incorporating into the food, feed or personal care product the beadlets obtained according to claim 1.

13. The process according to claim 12, which comprises incorporating 1 to 12 ppm of the at least one carotenoid into the food, feed and personal care product.

14. The process according to claim 12, wherein a food product is produced.

15. The process according to claim 14, wherein the food product is a soft drink.

16. The process according to claim 14, wherein the food product is a margarine.
